(19) United States
(12) Patent Application Publication
(10) Pub. No.: US 2010/0227870 A1
(43) Pub. Date: Sep. 9, 2010

(54) Novel Compounds

(76) Inventors: Massimo Gianotti, Verona (IT); Jag Paul Heer, Essex (GB); Peter John Lovell, Essex (GB); Catia Seri, Verona (IT); Ben Gerald Tehan, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/663,739

(22) PCT Filed: Jun. 12, 2008
(86) PCT No.: PCT/EP2008/057373

§ 371 Date: May 21, 2010

(30) Foreign Application Priority Data
Jun. 14, 2007 (GB) 0711521.5

(51) Int. Cl. A61K 31/4965; C07D 241/04; A61P 25/18

(52) U.S. Cl. 514/255.01; 544/391

(57) ABSTRACT

The invention provides a compound of formula (I) or a pharmaceutically acceptable salt thereof

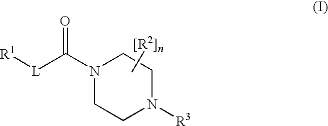

for treating diseases and conditions mediated by positive allosteric modulation of the G-protein coupled metabotropic subtype 5 receptor (mGluR5), such as neurological and psychiatric disorders, for example schizophrenia. In addition, the invention relates to compositions containing the derivatives and processes for their preparation.

The invention relates to piperazine derivatives for treating diseases and conditions mediated by positive allosteric modulation of the G-protein coupled metabotropic subtype 5 receptor (mGluR5), such as neurological and psychiatric disorders, for example schizophrenia. In addition, the invention relates to compositions containing the derivatives and processes for their preparation.

Glutamate is the major excitatory neurotransmitter in the nervous system and exerts its action through both ligand-gated ion channels and G-protein coupled metabotropic receptors (mGluR). Evidence suggests that mGlu5 receptors are generally located at post-synaptic levels and they are mainly distributed in limbic forebrain areas. Aberrant glutamatergic neurotransmission (due to excessive activation of glutamate receptors and abnormalities in glutamate receptor binding and mRNA expression), has been implicated in several neurological and psychiatric disorders. In particular, modulation of glutamatergic neurotransmission to alleviate a hypoglutamatergic state existing in schizophrenia has received increasing support over the past few years and may provide a complementary approach to traditional dopamine based therapies.

A number of mGluR5 agonists and antagonists have been identified to date which bind at the orthosteric binding site but suffer poor sub-type selectivity due to high sequence homology in this region. It has been suggested that positive allosteric modulation of mGluR5 may provide therapeutic benefits over orthosteric modulators for the treatment of neurological and psychiatric disorders such as schizophrenia (Kinney et al, J. Pharmacol. And Exp. Ther., (2005), 313 (1), 199-206; Lindsley et al, J. Med. Chem. (2004), 47 (24), 5825-8). Due to the conserved ligand binding domain within the mGluR family, allosteric modulators should provide greater selectivity over orthosteric modulators as the 7 transmembrane domain is less conserved between mGluR subtypes. In addition agonists binding to the orthosteric binding site have been suggested to be pro-convulsant (Chapman et al, Neuropharmacol. (2000), 39 (9), 1567-74), algesic (Walket et al, Neuropharmacol (2001), 40 (1), 1-9), neurotoxic (Blaabjerg et al (2001) 898 (1), 91-104) and anxiogenic (Perez de la Mora et al Eur. J. Neurosci. (2006) 23 (10), 2749-59), making allosteric modulation of the receptor a more attractive proposition. Allosteric modulators require the presence of an endogenous agonist and are saturable so should mimic the normal physiological effects of the endogenous ligand which suggests the effects of a patient taking an overdose of an allosteric modulator may be less than an overdose of an orthosteric modulator

It is therefore an object of the invention to provide compounds for treating diseases and conditions mediated by positive allosteric modulation of the G-protein coupled metabotropic subtype 5 receptor (mGluR5), such as neurological and psychiatric disorders, for example schizophrenia.

Therefore, according to a first aspect, the invention provides a compound of formula (I) or a pharmaceutically acceptable salt thereof

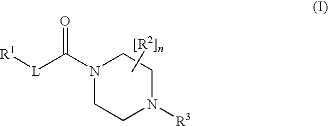

wherein
R¹ is phenyl optionally substituted by one or more halogens;
L is a three atom linker selected from the list consisting of —OC(R^{a})₂C(R^{b})₂—, and —C(═O)C(R^{a})₂C(R^{b})₂—, wherein the left hand atom of the linker is attached to R¹; wherein each R^{a} which may be the same or different, is hydrogen or C₁₋₃alkyl; or the two R^{a} groups together with the carbon atom to which they are attached form a 3-6-membered cycloalkane ring;
   each R^{b} which may be the same or different, is hydrogen or C₁₋₃alkyl; or the two R^{b} groups together with the carbon atom to which they are attached form a 3-6-membered cycloalkane ring; and
   wherein either the two R^{a} groups or the two R^{b} groups may form a cycloalkane ring;
n is 0, 1 or 2;
when n is 1 or 2, R² is independently C₁₋₃alkyl or haloC₁₋₃alkyl; and
R³ is phenyl optionally substituted by one or more groups independently selected from the list consisting of halogen, C₁₋₃alkyl, haloC₁₋₃alkyl, cyano, —CH(═NOH) and hydroxyC₁₋₃alkyl; wherein one of the groups is attached to the ortho position on the phenyl;
wherein the compound is not 4-oxo-1-phenyl-4-(4-phenyl-1-piperazinyl)-1-butanone;
1-(4-fluorophenyl)-4-oxo-4-(4-phenyl-1-piperazinyl)-1-butanone;
1-(4-fluorophenyl)-4-[4-(2-fluorophenyl)-1-piperazinyl]-4-oxo-1-butanone;
4-[4-(5-chloro-2-methylphenyl)-1-piperazinyl]-4-oxo-1-phenyl-1-butanone;
4-[4-(2-chlorophenyl)-1-piperazinyl]-4-oxo-1-phenyl-1-butanone; or
1-(2-methylphenyl)-4-[3-(phenyloxy)propanoyl]piperazine.

In a second aspect, the invention provides a compound of formula (I) or a pharmaceutically acceptable salt thereof

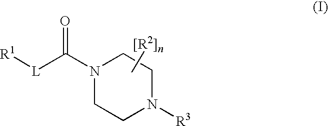

wherein
R¹ is phenyl optionally substituted by one or more halogens;
L is —OC(R^{a})₂C(R^{b})₂—, wherein the left hand atom of the linker is attached to R¹; wherein each R^{a} which may be the same or different, is hydrogen or C₁₋₃alkyl; or the two R^{a} groups together with the carbon atom to which they are attached form a 3-6-membered cycloalkane ring;
   each R^{b} which may be the same or different, is hydrogen or C₁₋₃alkyl; or the two R^{b} groups together with the carbon atom to which they are attached form a 3-6-membered cycloalkane ring; and
   wherein either the two R^{a} groups or the two R^{b} groups may form a cycloalkane ring;
n is 0, 1 or 2;
when n is 1 or 2, R² is independently C₁₋₃alkyl or haloC₁₋₃alkyl; and
R³ is phenyl optionally substituted by one or more groups independently selected from the list consisting of halogen, C₁₋₃alkyl, haloC₁₋₃alkyl, cyano, —CH(═NOH) and hydroxyC₁₋₃alkyl; wherein one of the groups is attached to the ortho position on the phenyl;
wherein the compound is not 1-(2-methylphenyl)-4-[3-(phenyloxy)propanoyl]piperazine.

The term “halogen” and its abbreviation “halo” refer to fluorine, chlorine, bromine or iodine. In an embodiment, unless otherwise indicated the halogen is fluorine or chlorine.

The term “C₁₋₃alkyl” refers to an alkyl group having from one to three carbon atoms, in any isomeric form. In an embodiment, unless otherwise indicated, the C₁₋₃alkyl group is selected from methyl, ethyl, propyl and isopropyl. Unless otherwise indicated, any alkyl group is straight or branched regardless of whether it forms part of another group, for example alkoxy and haloalkyl.

As used herein, a 3-6 membered cycloalkane ring comprises from 3 to 6 carbons all interconnected to form a ring. In an embodiment, unless otherwise indicated, the cycloalkane ring is selected from cyclopropyl, cyclobutyl, cyclopentyl and cyclohexyl.

The term “haloC₁₋₃alkyl” refers to a C₁₋₃alkyl group substituted with one or more halogens, for example —CF₃.

In an embodiment, L is a three atom linker selected from the list consisting of —OC(R^{a})₂C(R^{b})₂— and —C(═O)C(R^{a})₂C(R^{b})₂—, wherein the left hand atom of the linker is attached to R¹, wherein each R^{a} which may be the same or different, is hydrogen or C₁₋₃alkyl; and each R^{b} which may be the same or different, is hydrogen or C₁₋₃alkyl.

In an embodiment, L is a three atom linker selected from the list consisting of —OCH₂CH₂— and —C(═O)CH₂CH₂—, wherein the left hand atom of the linker is attached to R¹.

In an embodiment, L is —OCH₂CH₂—, wherein the left hand atom of the linker is attached to R¹.

In an embodiment, n is 0 or 1.

When n is 1, R² is methyl or —CF₃.

In a further embodiment n is 0.

In an embodiment, R³ is phenyl substituted by one, two or three groups independently selected from the list consisting of halogen, C₁₋₃alkyl, haloC₁₋₃alkyl, cyano, —CH(═NOH) and hydroxyC₁₋₃alkyl; one group being substituted at the ortho position by either cyano, —CH(═NOH) or hydroxyC₁₋₃alkyl.

In an embodiment, R³ is phenyl substituted by one or two groups independently selected from the list consisting of halogen, C₁₋₃alkyl, haloC₁₋₃alkyl, cyano, —CH(═NOH), and hydroxyC₁₋₃alkyl; one group being substituted at the ortho position by either cyano, —CH(═NOH) or hydroxyC₁₋₃alkyl.

In an embodiment, R³ is phenyl substituted by one or two groups independently selected from the list consisting of halogen, C₁₋₃alkyl, haloC₁₋₃alkyl, cyano, —CH(═NOH), and hydroxyC₁₋₃alkyl; one group being substituted at the ortho position by cyano.

In an embodiment, R³ is phenyl substituted at the ortho position by either cyano, —CH(═NOH) or hydroxyC₁₋₃alkyl.

In a further embodiment, the compound of formula (I) is selected from the list consisting of:
2-(4-{3-[(4-fluorophenyl)oxy]propanoyl}-1-piperazinyl)-6-(trifluoromethyl)benzonitrile;
2,3-dichloro-6-(4-{3-[(4-fluorophenyl)oxy]propanoyl}-1-piperazinyl)benzonitrile;
2-(4-{3-[(4-fluorophenyl)oxy]propanoyl}-1-piperazinyl)-5-(trifluoromethyl)benzonitrile;
3-chloro-6-(4-{3-[(4-fluorophenyl)oxy]propanoyl}-1-piperazinyl)-2-(trifluoromethyl)benzonitrile;
1-(2,3-dichloro-4-fluorophenyl)-4-{3-[(4-fluorophenyl)oxy]propanoyl}piperazine;
2-chloro-3-fluoro-6-(4-{3-[(4-fluorophenyl)oxy]propanoyl}-1-piperazinyl)benzonitrile;
2-(4-{3-[(4-fluorophenyl)oxy]propanoyl}-1-piperazinyl)-6-(trifluoromethyl)benzaldehyde;
2-fluoro-6-(4-{3-[(4-fluorophenyl)oxy]propanoyl}-1-piperazinyl)benzonitrile;
3,5-dichloro-2-(4-{3-[(4-fluorophenyl)oxy]propanoyl}-1-piperazinyl)benzonitrile;
1-[2-chloro-3-(trifluoromethyl)phenyl]-4-{3-[(4-fluorophenyl)oxy]propanoyl}piperazine;
2-{-4-[3-(phenyloxy)propanoyl]-1-piperazinyl}-6-(trifluoromethyl)benzonitrile;
1-(2,3-dichlorophenyl)-4-{3-[(4-fluorophenyl)oxy]propanoyl}piperazine;
5-fluoro-2-(4-{3-[(4-fluorophenyl)oxy]propanoyl}-1-piperazinyl)benzonitrile;
2-(4-{3-[(4-fluorophenyl)oxy]propanoyl}-1-piperazinyl)-6-(trifluoromethyl)benzaldehyde oxime;
2-(4-{3-[(4-fluorophenyl)oxy]propanoyl}-1-piperazinyl)-6-(trifluoromethyl)benzaldehyde O-methyloxime;
[2-(4-{3-[(4-fluorophenyl)oxy]propanoyl}-1-piperazinyl)-6-(trifluoromethyl)phenyl]methanol;
1-(2,3-dichlorophenyl)-4-[3-(phenyloxy)propanoyl]piperazine;
2-(4-{3-[(3-bromophenyl)oxy]propanoyl}-1-piperazinyl)-6-(trifluoromethyl)benzonitrile;
5-chloro-2-(4-{3-[(4-fluorophenyl)oxy]propanoyl}-1-piperazinyl)benzonitrile;
2-chloro-6-(4-{3-[(4-fluorophenyl)oxy]propanoyl}-1-piperazinyl)benzonitrile;
5-fluoro-2-{-4-[3-(phenyloxy)propanoyl]-1-piperazinyl}benzonitrile;
1-{3-[(4-bromophenyl)oxy]propanoyl}-4-(2,3-dichlorophenyl)piperazine;
1-(2,3-dichlorophenyl)-4-{3-[(3-fluorophenyl)oxy]propanoyl}piperazine;
2-[4-(4-oxo-4-phenylbutanoyl)-1-piperazinyl]-6-(trifluoromethyl)benzonitrile;
2-(4-{3-[(4-fluorophenyl)oxy]propanoyl}-1-piperazinyl)benzonitrile;
1-{3-[(4-fluorophenyl)oxy]propanoyl}-4-[4-(trifluoromethyl)phenyl]piperazine;
2-{4-[3-(phenyloxy)propanoyl]-1-piperazinyl}benzonitrile;
1-(2-chlorophenyl)-4-[3-(phenyloxy)propanoyl]piperazine;
1-(2,6-dimethylphenyl)-4-[3-(phenyloxy)propanoyl]piperazine;
4-[4-(2,3-dichlorophenyl)-1-piperazinyl]-1-(4-fluorophenyl)-4-oxo-1-butanone;
1-(2,3-dimethylphenyl)-4-[3-(phenyloxy)propanoyl]piperazine;
1-(2,3-dichlorophenyl)-4-{3-[(3,4-difluorophenyl)oxy]propanoyl}piperazine; and
2-(4-{3-[(3-fluorophenyl)oxy]propanoyl}-1-piperazinyl)-6-(trifluoromethyl)benzonitrile;
   or a pharmaceutically acceptable salt thereof.

For the avoidance of doubt, unless otherwise indicated, the term substituted means substituted by one or more defined groups. In the case where groups may be selected from a number of alternative groups, the selected groups may be the same or different.

For the avoidance of doubt, the term independently means that where more than one substituent is selected from a number of possible substituents, those substituents maybe the same or different.

The compounds of formula (I) may form pharmaceutically or veterinarily acceptable salts, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

Hereinafter, the compounds of formula (I) and their pharmaceutically acceptable salts, are referred to as “the compounds of the invention”.

It will be appreciated by those skilled in the art that certain protected derivatives of the compounds of the invention, which may be made prior to a final deprotection stage, may not possess pharmacological activity as such, but may, in certain instances, be administered orally or parenterally and thereafter metabolised in the body to form compounds defined in the first aspect which are pharmacologically active. Such derivatives may therefore be described as “prodrugs”. All protected derivatives and prodrugs of compounds defined in the first aspect are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within the compound defined in the first aspect.

The compounds of the invention may exist in solvated or hydrated form.

The compounds of the invention or solvates/hydrates of the compounds or salts, may exist in one or more polymorphic forms.

Therefore, according to a further aspect, the invention provides a solvate, hydrate or prodrug of the compounds of the invention.

Certain compounds of the invention may exist in one or more tautomeric forms. All tautomers and mixtures thereof are included in the scope of the present invention. For example, a claim to 2-hydroxypyridyl would also cover its tautomeric form, α-pyridinonyl.

Certain compounds of the invention possess one or more chiral centres and so exist in a number of stereoisomeric forms. Compounds having one chiral centre may exist as enantiomers or a racemic mixture containing enantiomers. Compounds having two or more chiral centres may exist as diastereoismomers or enantiomers. All sterioisomers (for example enantiomers and diastereoisomers) and mixtures thereof are included in the scope of the present invention. Racemic mixtures may be separated to give their individual enantiomer using preparative HPLC using a column with a chiral stationary phase or resolved to yield individual enantiomers utilising methods known to those skilled in the art. In addition, chiral intermediate compounds may be resolved and used to prepare individual enantiomers.

The invention also includes all suitable isotopic variations of the compounds of the invention. An isotopic variation of the compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, sulphur, fluorine and chlorine such as ²H, ³H, ¹³C, ¹⁴C, ¹⁵N, ¹⁷O, ¹⁸O, ³⁵S, ¹⁸F and ³⁶Cl respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

Compounds of the invention may be prepared in a variety of ways. In the following reaction schemes and hereinafter, unless otherwise stated R¹ to R³, L and n are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic), etc. . . . (IVa), (IVb), (IVc) etc.

Compounds of formula (I) may be prepared according to reaction scheme 1 by reacting compounds of formula (II) with compounds of formula (III). Typical reaction conditions comprise reacting (II) with a suitable amide coupling reagent (eg HATU or EDC/HOBt) and (III) in the presence of a suitable base (eg DIPEA) in an aprotic solvent (eg DMF, dioxan) at room temperature with a reaction time of 1-2 days.

Compounds of formula (II) are either commercially available or may be prepared by procedures known to the skilled person.

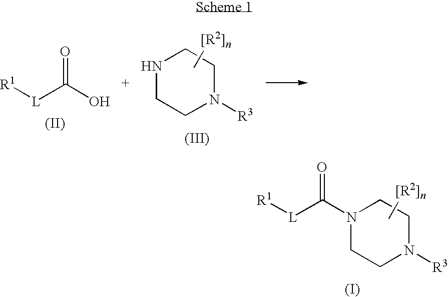

Compounds of formula (III) where R³ is substituted phenyl, may be prepared according to reaction scheme 2 by reacting compounds of formula (IV) and compounds of formula (V) where X is halogen (eg fluoro or chloro) in an aprotic solvent (eg NMP) at an elevated temperature (eg 200° C.) using a microwave reactor for approximately 15 minutes.

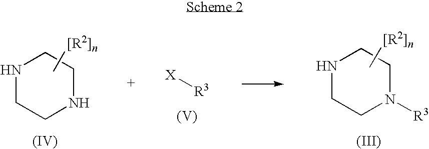

Compounds of formula (IIIa), i.e. compounds of formula (III) where R³ is phenyl substituted by at least one electron withdrawing group (eg CN) at an ortho position, may be prepared according to reaction scheme 3 by reacting compounds of formula (IV) with compounds of formula (Va) in an aprotic solvent (eg DMF) at an elevated temperature (eg 90° C.) for approximately 4 hrs.

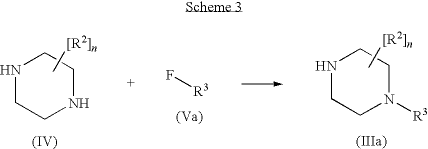

Alternatively, compounds of formula (I) may be prepared according to reaction scheme 4 by reacting compounds of formula (VI) and compounds of formula (V) where X is halogen, in the presence of a suitable base (eg DIPEA) in an aprotic solvent (eg DMF) at an elevated temperature (eg 180° C.) for approximately 45 mins.

Compounds of formula (V) are either commercially available or may be prepared by procedures known to the skilled person.

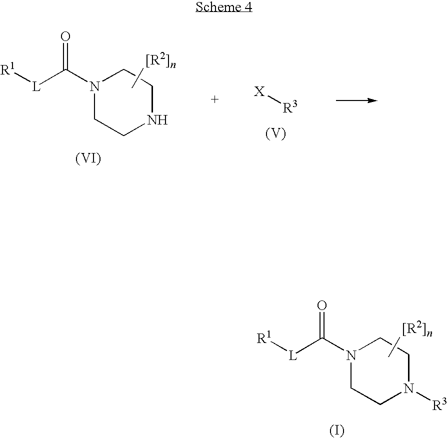

Compounds of formula (VI) may be prepared in two steps according to reaction scheme 5. Firstly compounds of formula (II) are reacted with a suitable amide coupling reagent (eg EDC/HOBt) and compounds of formula (VII) in an aprotic solvent (eg dioxan) at room temperature for 12 hrs to give compounds of formula (VIII). The BOC protecting group may be removed by reacting compounds of formula (VIII) with a bronsted acid (eg HCl) in an aprotic solvent (eg dioxan) at room temperature for 2 hrs.

Compounds of formula (VII) are either commercially available or may be prepared by procedures known to the skilled person.

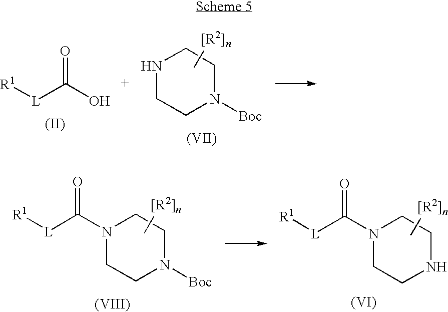

As discussed above, it is considered that positive allosteric modulation of mGluR5 may treat neurological and psychiatric disorders, for example schizophrenia.

Therefore, according to an embodiment, the invention provides a compound of the invention for use as a medicament, preferably a human medicament.

According to a further aspect, the invention provides the use of a compound of the invention in the manufacture of a medicament for treating or preventing a disease or condition mediated by positive allosteric modulation of mGluR5.

In an embodiment, diseases or conditions that may be mediated by positive allosteric modulation of mGluR5 are selected from the list consisting of: [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Psychotic disorders for example Schizophrenia (including the subtypes Paranoid Type (295.30), Disorganised Type (295.10), Catatonic Type (295.20), Undifferentiated Type (295.90) and Residual Type (295.60)); Schizophreniform Disorder (295.40); Schizoaffective Disorder (295.70) (including the subtypes Bipolar Type and Depressive Type); Delusional Disorder (297.1) (including the subtypes Erotomanic Type, Grandiose Type, Jealous Type, Persecutory Type, Somatic Type, Mixed Type and Unspecified Type); Brief Psychotic Disorder (298.8); Shared Psychotic Disorder (297.3); Psychotic Disorder due to a General Medical Condition (including the subtypes with Delusions and with Hallucinations); Substance-Induced Psychotic Disorder (including the subtypes with Delusions (293.81) and with Hallucinations (293.82)); and Psychotic Disorder Not Otherwise Specified (298.9).
ii) Anxiety disorders for example Social Anxiety Disorder; Panic Attack; Agoraphobia, Panic Disorder; Agoraphobia Without History of Panic Disorder (300.22); Specific Phobia (300.29) (including the subtypes Animal Type, Natural Environment Type, Blood-Injection-Injury Type, Situational Type and Other Type); Social Phobia (300.23); Obsessive-Compulsive Disorder (300.3); Posttraumatic Stress Disorder (309.81); Acute Stress Disorder (308.3); Generalized Anxiety Disorder (300.02); Anxiety Disorder Due to a General Medical Condition (293.84); Substance-Induced Anxiety Disorder; and Anxiety Disorder Not Otherwise Specified (300.00).
iii) Sleep disorders for example primary sleep disorders such as Dyssomnias (including Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47)); primary sleep disorders such as Parasomnias (including Nightmare Disorder (307.47), Sleep Terror Disorder (307.46), Sleepwalking Disorder (307.46) and Parasomnia Not Otherwise Specified (307.47)); Sleep Disorders Related to Another Mental Disorder (including Insomnia Related to Another Mental Disorder (307.42) and Hypersomnia Related to Another Mental Disorder (307.44)); Sleep Disorder Due to a General Medical Condition; and Substance-Induced Sleep Disorder (including the subtypes Insomnia Type, Hypersomnia Type, Parasomnia Type and Mixed Type).
iv) Attention-Deficit/Hyperactivity Disorder (including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9)); Hyperkinetic Disorder; Disruptive Behaviour Disorders such as Conduct Disorder (including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).
v) Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).
vi) Enhancement of cognition including the treatment of cognition impairment in other diseases such as schizophrenia, bipolar disorder, depression, other psychiatric disorders and psychotic conditions associated with cognitive impairment, e.g. Alzheimer's disease.

It will be appreciated that references herein to “treatment” extend to prophylaxis, prevention of recurrence and suppression or amelioration of symptoms (whether mild, moderate or severe) as well as the treatment of established conditions. The compound of the invention may be administered as the raw chemical but the active ingredient is suitably presented as a pharmaceutical formulation.

The compounds of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The compounds of the invention may be used in combination with the following agents to treat or prevent bipolar disease: i) mood stabilisers; ii) antipsychotics; and iii) antidepressants.

The compounds of the invention may be used in combination with the following agents to treat or prevent anxiety disorders: i) anxiolytics; and ii) antidepressants.

Antipsychotic drugs include Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone and amisulpride).

Antidepressant drugs include serotonin reuptake inhibitors (such as citalopram, escitalopram, fluoxetine, paroxetine, sertraline femoxetine, fluvoxamine, indalpine and zimeldine); dual serotonin/noradrenaline reuptake inhibitors (such as venlafaxine, duloxetine and milnacipran); Noradrenaline reuptake inhibitors (such as reboxetine and venlafaxine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabiliser drugs include lithium, sodium valproate/valproic acid/divalproex, carbamazepine, lamotrigine, gabapentin, topiramate and tiagabine.

Anxiolytics include benzodiazepines such as alprazolam and lorazepam.

In addition the compounds of the invention may be administered in combination with 5-HT₃ antagonists (such as ondansetron, granisetron and metoclopramide); serotonin agonists (such as sumatriptan, rauwolscine, yohimbine and metoclopramide); and NK-1 antagonists.

It will be appreciated that the compound of the combination or composition may be administered simultaneously (either in the same or different pharmaceutical formulations), separately or sequentially.

It will be appreciated that references herein to “treatment” extend to prophylaxis, prevention of recurrence and suppression or amelioration of symptoms (whether mild, moderate or severe) as well as the treatment of established conditions.

The compounds of the invention will normally, but not necessarily, be formulated into pharmaceutical compositions prior to administration to a patient by an appropriate route. Accordingly, in another aspect, the invention provides pharmaceutical compositions comprising a compound of the invention and one or more pharmaceutically-acceptable excipients.

As used herein, “pharmaceutically-acceptable excipient” means any pharmaceutically acceptable material present in the pharmaceutical composition or dosage form other than the compound or compounds of the invention. Typically the material gives form, consistency and performance to the pharmaceutical composition.

The pharmaceutical compositions of the invention typically contain one compound of the invention. However, in certain embodiments, the pharmaceutical compositions of the invention contain more than one compound of the invention. In addition, the pharmaceutical compositions of the invention may comprise one or more additional pharmaceutically active compounds.

Such pharmaceutical compositions of the invention may be prepared and packaged in bulk form wherein a safe and effective amount of a compound of the invention can be dispensed and then given to the patient such as with powders or syrups. Alternatively, the pharmaceutical compositions of the invention may be prepared and packaged as dosage forms wherein each physically discrete dosage form contains a safe and effective amount of a compound of the invention. Accordingly, in another aspect, the invention provides dosage forms comprising pharmaceutical compositions of the invention. Each discrete dosage form contains from 1 mg to 500 mg of a compound of the invention. In another aspect, each discrete dosage form contains from 5 mg to 400 mg of a compound of the invention. In another aspect, each discrete dosage form contains from 10 mg to 300 mg of a compound of the invention. In another aspect, each discrete dosage form contains from 20 mg to 300 mg of a compound of the invention.

It will be recognised by one of skill in the art that the optimal quantity and spacing of individual dosages of compounds of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of compounds of the invention given per day for a defined number of days, can be ascertained by those skilled in the art using conventional course of treatment determination tests.

The compositions of the invention will typically be formulated into dosage forms which are adapted for administration to the patient by the desired route of administration. For example, dosage forms include those adapted for (1) oral administration such as tablets, capsules, caplets, pills, lozenges, powders, syrups, elixirs, suspensions, solutions, emulsions, sachets and cachets; (2) parenteral administration such as sterile solutions, suspensions, implants and powders for reconstitution; (3) transdermal administration such as transdermal patches; (4) rectal and vaginal administration such as suppositories, pessaries and foams; (5) inhalation and intranasal such as dry powders, aerosols, suspensions and solutions (sprays and drops); (6) topical administration such as creams, ointments, lotions, solutions, pastes, drops, sprays, foams and gels; (7) ocular administration such as drops, ointment, sprays, suspensions and inserts; (8) buccal and sublingual administration such as lozenges, patches, sprays, drops, chewing gums and tablets.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance. Certain pharmaceutically-acceptable excipients may be chosen for their ability to facilitate the release of the compound of the invention at the appropriate rate to treat the condition.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavouring agents, flavour masking agents, colouring agents, anticaking agents, humectants, chelating agents, plasticizers, viscosity increasing agents, rate modifying agents, antioxidants, preservatives, stabilizers, surfactants and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to determine suitable pharmaceutically-acceptable excipients in appropriate amounts for use with the compounds of the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press). The pharmaceutical compositions of the invention may be prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch and pre-gelatinized starch), gelatin, acacia, sodium alginate, alginic acid, tragacanth, guar gum, povidone, and cellulose and its derivatives (e.g. hydroxypropyl methyl cellulose). The oral solid dosage form may further comprise a disintegrant. Suitable disintegrants include starches, crospovidone, sodium starch glycolate, cros-carmellose, alginic acid, and sodium carboxymethyl cellulose. The oral solid dosage form may further comprise a lubricant. Suitable lubricants include stearic acid, magnesium stearate, calcium stearate, and sodium dodecyl sulphate. The oral solid dosage form may further comprise a glidant such as talc and colloidal silicon dioxide. The oral solid dosage form may further comprise an outer coating which may have cosmetic or functional properties.

It will be appreciated that the invention includes the following further aspects. The diseases and conditions described above extend, where appropriate, to these further aspects.
i) A compound of the invention for use in treating or preventing a disease or condition mediated by positive allosteric modulation of mGluR5.
ii) A method of treatment or prevention of a disease or condition mediated by positive allosteric modulation of mGluR5 in a mammal comprising administering an effective amount of a compound of the invention.

### Supporting Compounds and Intermediates

The invention is supported by the following compounds described below.

In the procedures that follow, after each starting material, reference to an intermediate or compound is often provided. This is provided merely for assistance to the skilled chemist. The starting material may not necessarily have been prepared from the batch referred to.

Compounds are named using ACD/Name PRO 6.02 chemical naming software (Advanced Chemistry Development Inc., Toronato, Ontario, M5H2L3, Canada).

Proton Magnetic Resonance (NMR) spectra were recorded either on Varian instruments at 300, 400 or 500 MHz, or on a Bruker instrument at 300 or 400 MHz. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is reported.

Mass spectra (MS) were taken on a 4 II triple quadrupole Mass Spectrometer (Micromass UK) or on a Agilent MSD 1100 Mass Spectrometer, operating in ES (+) and ES (−) ionization mode or on a Agilent LC/MSD 1100 Mass Spectrometer, operating in ES (+) and ES (−) ionization mode coupled with HPLC instrument Agilent 1100 Series [LC/MS−ES (+): analysis performed on an Waters Atlantis column (50×4.6 mm); stationary phase particle size 3 uM; mobile phase A: aqueous phase=water+0.05% formic acid; mobile phase B=organic solvent=acetonitrile+0.05% formic acid. Method as follows:

| Time/min | % A | % B |
|---|---|---|
| 0 | 97 | 3 |
| 0.1 | 97 | 3 |
| 4 | 3 | 97 |
| 4.8 | 3 | 97 |
| 4.9 | 97 | 3 |
| 5.0 | 97 | 3 |

The above method has a flow rate of 3 mL/min. The injection volume is 5 μL. The column temperature is 30 degC. The UV detection range is from 220 to 330 nm.

For reactions involving microwave irradiation, a Biotage Initiator was used.

Flash silica gel chromatography was carried out on silica gel 230-400 mesh (supplied by Merck AG Darmstadt, Germany) or over pre-packed Biotage silica cartridges.

SPE-SCX cartridges were supplied by Varian. The eluent used with SPE-SCX cartridges was methanol followed by 2N ammonia solution in methanol.

In a number of preparations purification was performed using either Biotage manual flash chromatography (Flash+) or automatic flash chromatography (Horizon) systems. All these instruments work with Biotage Silica cartridge.

SPE-Si cartridges were supplied by Varian.

### Abbreviations

The following table lists the used abbreviations:
BINAP—2,2′-Bis(diphenylphosphino)-1,1′-binaphthalene
BOC—tert Butoxycarbonyl
DCM—Dichloromethane
DIPEA—Diisopropylethylamine
DMF—N,N-dimethylformamide
DMSO Dimethylsulphoxide
EDC—1-Ethyl-3-[3-dimethylaminopropyl]carbodiimide hydrochloride
HATU—(O-7-azabenzotriazol-1-yl)-N,N,N′,N′-tetramethyluronium hexafluorophosphate)
HOAt—1-Hydroxy-7-azabenzotriazole
HOBt—1-Hydroxybenzotriazole
NMP—N-Methylpyrrolidinone
Pd₂ dba₃—Tris(dibenzylideneacetone)dipalladium(0)
THF—Tetrahydrofuran

### Intermediate 1: 2-(1-Piperazinyl)-6-(trifluoromethyl)benzonitrile

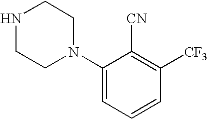

To a stirred solution of 2-fluoro-6-(trifluoromethyl)benzonitrile (19.6 g, 104 mmol) (Aldrich) in DMF (75 mL) at room temperature under an atmosphere of argon was added piperazine (26.8 g, 311 mmol, 3 equivalents). The reaction was heated to 90° C. for 4 hrs and 5 minutes and then concentrated under reduced pressure. The residue was partitioned between saturated aqueous NaHCO₃ and DCM and the organic layer dried over Na₂SO₄ and concentrated. The residue was purified by flash chromatography eluting with 2% MeOH/DCM to 10% MeOH/DCM to afford the title compound (21.3 g); m/z: 256 [M+H]⁺, retention time 1.75 mins.

### Intermediate 2: 2-Fluoro-6-(1-piperazinyl)benzonitrile

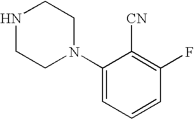

2,6 Difluorobenzonitrile (0.42 g, 3.03 mmol) (Aldrich) and piperazine (1.28 g, 14.9 mmol) in DMF (4 mL) were heated at 80° C. for 3.5 hrs. Upon cooling, the reaction mixture was partitioned between DCM, water and aqueous saturated sodium bicarbonate. The organic phase was dried over Na₂SO₄ and filtered and the solvent evaporated to give a yellow gum. Purification on silica gel afforded the title compound (335 mg); m/z: 206 [M+H]⁺, retention time 1.52 mins.

### Intermediate 3: 1-[2-Chloro-4-(trifluoromethyl)phenyl]piperazine

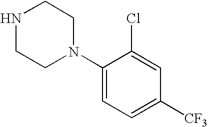

2-Chloro-1-fluoro-4-(trifluoromethyl)benzene (300 mg, 1.5 mmol) (FluoroChem) and piperazine (261 mg, 3.0 mmol) dissolved in NMP (1 mL) were heated in a microwave reactor at 200° C. for 15 minutes. The reaction mixture was partitioned between DCM and water and the organic layer passed through an SCX column. The crude material was purified by flash chromatography on silica eluting with 1% MeOH/DCM to 5% MeOH/DCM to afford the title compound as a white solid (380 mg); m/z: 265/267 [M+H]⁺, retention time 1.82 mins.

The following compounds of formula (IIIb), i.e. compounds of general formula (III) where n is 0 were prepared by a similar procedure to that described for the preparation of Intermediate 3 using piperazine and the halobenzene indicated.

| | |
|---|---|
| | (IIIb) |
| 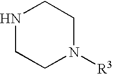 | |

| | | | | Retention |
|---|---|---|---|---|
| | | | m/z | time (min) |
| Intermediate | Halobenzene | R³ | [M + H]⁺ | 5 min LC/MS |
| 4 | 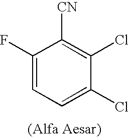 | 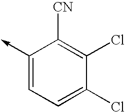 | 256, 258, 260 | 1.99 |
| 5 | 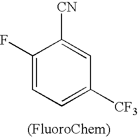 | 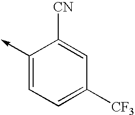 | 256 | 1.82 |
| 6 | 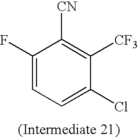 | 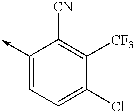 | 290, 292 | 1.71 |
| 7 | 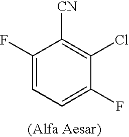 | 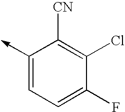 | 240, 242 | 1.42 |
| 8 | 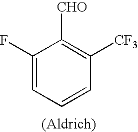 | 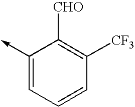 | 259 | 1.68 |
| 9 | 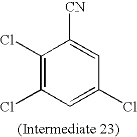 | 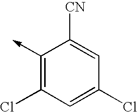 | 256, 258, 260 | 1.58 |
| 10 | 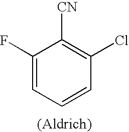 | 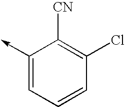 | 222, 224 | 1.41 |
| 11 | 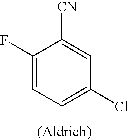 | 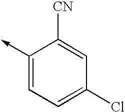 | 222, 224 | 1.43 |
| 12 | 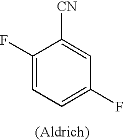 | 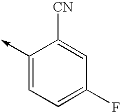 | 206 | 1.30 |

### Intermediate 13: 1,1-Dimethylethyl 4-(2,3-dichloro-4-fluorophenyl)-1-piperazine carboxylate

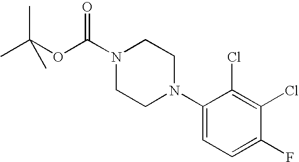

A mixture of 1-bromo-2,3-dichloro-4-fluorobenzene (1.23 g) (Aldrich), N-BOC-piperazine (1.03 g, 1.1 equivs), Pd₂(dba)₃ (92 mg, 2 mol %), BINAP (167 mg, 5 mol %) and NaOtBu (727 mg, 1.5 equivs) in toluene (15 mL) was heated to 140° C. for 1 hr using a microwave reactor. The reaction mixture was then diluted with EtOAc and washed with water and the organic phase further washed with bicarbonate solution and dried using Na₂SO₄. Concentration of the organic phase gave the crude product which was purified on silica to afford the title compound (3.77 g); ¹H NMR (CDCl₃): 7.04 (1H, m), 6.93 (1H, m), 3.60 (4H, brs), 2.93 (4H, brs), 1.49 (9H, s).

### Intermediate 14: 1-(2,3-Dichloro-4-fluorophenyl)piperazine

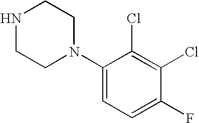

To a solution of 1,1-dimethylethyl 4-(2,3-dichloro-4-fluorophenyl)-1-piperazine carboxylate (Intermediate 13) (3.77 g) in ethanol (15 mL) was added 4M HCl in dioxan (14 mL, 5 equivs) and stirred at room temperature for 5 hrs. The solvents were removed and the residue triturated with ether to afford the title compound (3.02 g) as a hydrochloride salt; ¹H NMR (CDCl₃): 9.44 (1H, brs), 7.47 (1H, m), 7.29 (1H, m), 3.44-3.20 (8H, m).

### Intermediate 15: 2-Chloro-3-(trifluoromethyl)phenyl trifluoromethanesulfonate

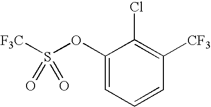

To a solution of 2-chloro-3-(trifluoromethyl)phenol (500 mg, 2.54 mmol) (Aldrich) in DCM (20 mL) at 0° C. under an argon atmosphere was added pyridine (246 μL, 3.05 mmol) and triflic anhydride (428 μL, 2.54 mmol). The mixture was allowed to warm to room temperature and stirred for 50 minutes. The reaction was quenched with water and extracted with DCM. The solvent was evaporated and the crude product purified on silica gel eluting with 10% ethylacetate/hexane to afford the title compound (447 mg); m/z: 206 [M−H]⁻, retention time 3.55 mins.

### Intermediate 16: 1,1-dimethylethyl 4-[2-chloro-3-(trifluoromethyl)phenyl]-1-piperazinecarboxylate

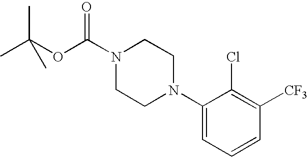

To a solution of 2-chloro-3-(trifluoromethyl)phenyl trifluoromethanesulfonate (Intermediate 15) (247 mg, 0.75 mmol) in dry toluene (15 mL) under an atmosphere of argon was added N-BOC-piperazine (168 mg, 0.9 mmol), Pd₂(dba)₃ (172 mg, 0.18 mmol) and sodium tert-butoxide (101 mg, 1.05 mmol). The reaction mixture was heated to 80° C. for 7 hrs, cooled and then diluted with ethyl acetate. Celite was added and the resulting mixture was stirred for 15 mins before the celite was removed by filtration and the solution passed through a pad of silica, washing with ethyl acetate. The organic solvents were removed by evaporation under reduced pressure and the residue purified on silica gel chromatography to afford the title compound which was not characterised but taken straight onto the preparation of Intermediate 17.

### Intermediate 17: 1-[2-Chloro-3-(trifluoromethyl)phenyl]piperazine

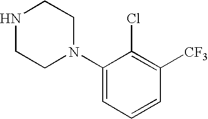

To a solution of 1,1-dimethylethyl 4-[2-chloro-3-(trifluoromethyl)phenyl]-1-piperazinecarboxylate (Intermediate 16) (76 mg) in DCM (3 mL) at room temperature was added TFA (0.5 mL) slowly and the reaction mixture stirred for 2 hrs. Methanol was added and the reaction mixture was passed through SCX to afford the title compound (42 mg); m/z: 265/267 [M+H]⁺, retention time 1.98 mins.

### Intermediate 18: 1,1-Dimethylethyl 4-{3-[(4-fluorophenyl)oxy]propanoyl}-1-piperazine carboxylate

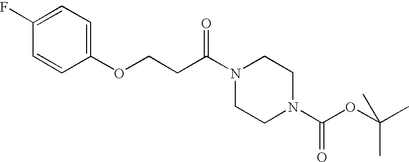

To a solution of 4-fluorophenoxypropionic acid (2.45 g, 13 mmol) (Aldrich), EDC hydrochloride (3.85 g, 20 mmol) and HOBt (1.8 g, 13 mmol) in dioxan (40 mL) was added BOC piperazine (2.5 g, 13 mmol) portionwise over 2 mins at room temperature. The reaction mixture was stirred at room temperature for 12 hrs and then concentrated. The residue was partitioned between ethyl acetate and water and the organic phase washed with 1N NaOH, then water and dried over Na₂SO₄. The organic phase was concentrated to afford the title compound as a white solid (4.58 g); m/z: 353 [M+H]⁺, retention time 2.94 mins.

### Intermediate 19: 1-{3-[(4-fluorophenyl)oxy]propanoyl}piperazine hydrochloride

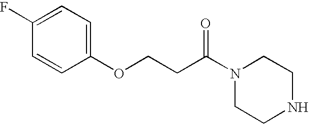

A solution of 1,1-dimethylethyl 4-{3-[(4-fluorophenyl)oxy]propanoyl}-1-piperazine carboxylate (Intermediate 18) (4.58 g, 13 mmol) in 4N HCl in dioxan (15 mL, 60 mmol) was stirred at room temperature under argon for 2 hrs. The resulting precipitate was collected by filtration and washed thoroughly with diethyl ether (50 mL). The solid was dried in vacuo to afford the title compound (3.65 g); ¹H NMR (d6 DMSO): 9.45 (1H, brs), 7.11 (2H, m), 6.94 (2H, m), 4.18 (2H, m), 3.74 (4H, m), 3.07 (4H, m), 2.85 (2H, m).

### Intermediate 20: 3-Chloro-6-fluoro-2-(trifluoromethyl)benzaldehyde oxime

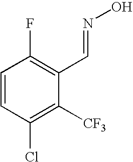

To a solution of 3-chloro-6-fluoro-2-(trifluoromethyl)benzaldehyde (ChemPur GmbH) (2.0 g, 8.9 mmol) in ethanol (50 mL) was added hydroxylamine hydrochloride (922 mg, 13.3 mmol, 1.5 equiv). The reaction mixture was stirred at room temperature for 5.5 hrs before the solvents were removed and the crude product partitioned between ethyl acetate and water. The organic phase was separated and dried over sodium sulphate. The crude product was purified on silica gel eluting with ethyl acetate/hexane to afford the title compound as a mixture of two isomers (1.0 g). m/z: 242, 244 [M+H]⁺, retention time 2.67 mins.

### Intermediate 21: 3-Chloro-6-fluoro-2-(trifluoromethyl)benzonitrile

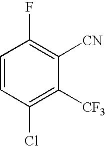

A solution of 3-chloro-6-fluoro-2-(trifluoromethyl)benzaldehyde oxime (Intermediate 20, 1.0 g, 4.2 mmol) in acetic anhydride (6 mL) was heated to 100° C. under argon for 3 hrs. The solution was then partitioned between ethyl acetate and water. The organic phase was separated, dried over sodium sulphate and concentrated in vacuo. The residue was purified on silica gel eluting with ethyl acetate/hexane to afford the title compound (366 mg). m/z: 224, 226 [M+H]⁺, retention time 3.53 mins.

### Intermediate 22: 2,3,5-Trichlorobenzaldehyde oxime

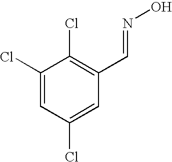

To a solution of 2,3,5-trichlorobenzaldehyde (Aldrich) (1.0 g, 4.8 mmol) in ethanol (20 mL) was added hydroxylamine hydrochloride (501 mg, 7.2 mmol) and the mixture stirred at room temperature for 165 minutes. The organic solvents were removed and the crude product was partitioned between ethyl acetate and water. The organic phase was separated, dried over sodium sulphate and concentrated in vacuo to afford the title compound (1.14 g) which was used without further purification.

### Intermediate 23: 2,3,5-Trichlorobenzonitrile

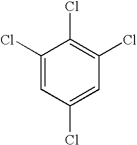

A solution of 2,3,5-trichlorobenzaldehyde oxime (Intermediate 22, 1.14 g, 4.8 mmol) in acetic anhydride (10 mL) was heated to reflux for 3.5 hrs. The solution was then partitioned between ethyl acetate and water. The organic phase was separated, dried over sodium sulphate and concentrated in vacuo. The residue was purified on silica gel eluting to afford the title compound (592 mg), ¹H NMR (CDCl₃): 7.71 (1H, d, 2.3 Hz), 7.60 (1H, d, 2.3 Hz) contaminated with 1-[bis(ethyloxy)methyl]-2,3,5-trichlorobenzene.

### Compound 1: 2-(4-{3-[(4-Fluorophenyl)oxy]propanoyl}-1-piperazinyl)-6-(trifluoromethyl)benzonitrile

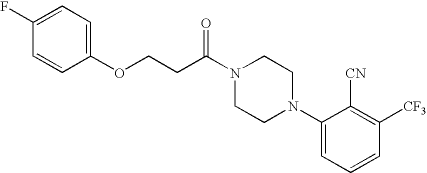

To a mixture of 2-(1-piperazinyl)-6-(trifluoromethyl)benzonitrile (Intermediate 1) (1.0 g, 3.9 mmol), 3-[(4-fluorophenyl)oxy]propanoic acid (Aldrich) (906 mg, 3.9 mmol) and DIPEA (1.4 mL, 7.8 mmol) in DMF (30 mL) was added HATU (1.49 g, 3.9 mmol) slowly. The solution was stirred at room temperature for 16 hrs and then quenched by the addition of saturated aqueous NaHCO₃ and extracted with DCM. The crude material was purified by flash chromatography on silica eluting with 50% ethyl acetate/hexane to afford the title compound as a white solid (1.51 g); m/z: 422 [M+H]⁺, retention time 3.23 mins.

The following compounds of formula (Ia), i.e. compounds of general formula (I) where n is 0, were prepared using a similar procedure to that described in the preparation of Compound 1 using the appropriate piperazine intermediate or commercially available piperazine and appropriate commercially available carboxylic acid (Aldrich).

| | |
|---|---|
| | (Ia) |
| 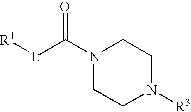 | |

| | | | | | Retention |
|---|---|---|---|---|---|
| | | | | | time (min) |
| | | | | m/z | 5 min |
| Cmp | R¹ | L | R³ | [M + H]⁺ | LC/MS |
| 2 | 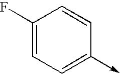 | —OCH₂CH₂— | 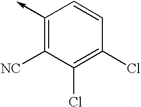 | 422, 424, 426 | 3.38 |
| 3 | 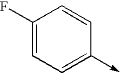 | —OCH₂CH₂— | 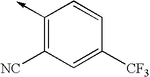 | 422 | 3.30 |
| 4 | 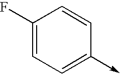 | —OCH₂CH₂— | 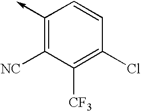 | 456, 458 | 3.39 |
| 5 | 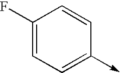 | —OCH₂CH₂— | 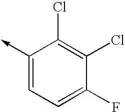 | 415, 417, 419 | 3.53 |
| 6 | 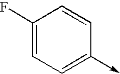 | —OCH₂CH₂— | 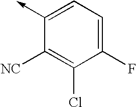 | 406, 408 | 3.15 |
| 7 | 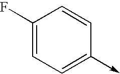 | —OCH₂CH₂— | 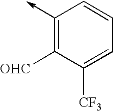 | 425 | 3.31 |
| 8 | 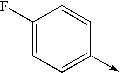 | —OCH₂CH₂— | 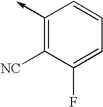 | 372 | 3.13 |
| 9 | 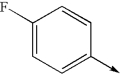 | —OCH₂CH₂— | 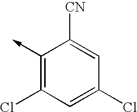 | 422 | 3.52 |
| 10 | 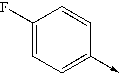 | —OCH₂CH₂— | 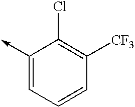 | 431, 433 | 3.52 |
| 11 | 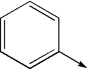 | —OCH₂CH₂— | 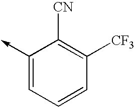 | 404 | 3.26 |
| 12 | 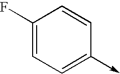 | —OCH₂CH₂— | 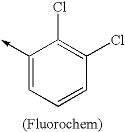 | 397, 399, 401 | 3.49 |
| 13 | 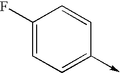 | —OCH₂CH₂— | 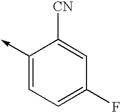 | 372 | 3.12 |

### Compound 14: 2-(4-{3-[(4-Fluorophenyl)oxy]propanoyl}-1-piperazinyl)-6-(trifluoromethyl)benzaldehyde oxime

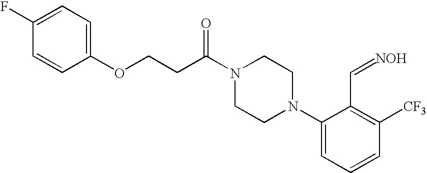

To a suspension of 2-(4-{3-[(4-fluorophenyl)oxy]propanoyl}-1-piperazinyl)-6-(trifluoromethyl)benzaldehyde (Compound 7) (100 mg, 0.24 mmol) in ethanol (5 mL) was added hydroxylamine hydrochloride (20 mg, 0.28 mmol) and sodium acetate (29 mg, 0.35 mmol). The suspension dissolved to give a clear solution which then became cloudy. After 3 hrs the solvent was evaporated and the residue was dissolved in water and extracted with DCM. The organic layer was dried and again evaporated. The residue was purified by trituration with ethyl acetate to give the title compound as a white solid (79 mg); m/z: 440 [M+H]⁺, retention time 2.94 mins.

### Compound 15: 2-(4-{3-[(4-Fluorophenyl)oxy]propanoyl}-1-piperazinyl)-6-(trifluoromethyl)benzaldehyde O-methyloxime

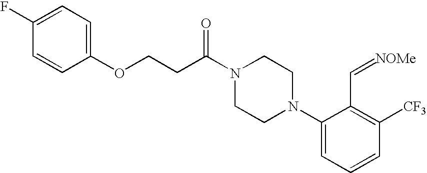

The title compound was prepared from 2-(4-{3-[(4-fluorophenyl)oxy]propanoyl}-1-piperazinyl)-6-(trifluoromethyl)benzaldehyde (Compound 7) and hydroxylamine methyl ether using a procedure similar to that described for the preparation of Compound 14; 71 mg; m/z: 454 [M+H]⁺, retention time 3.35 mins.

### Compound 16: [2-(4-{3-[(4-Fluorophenyl)oxy]propanoyl}-1-piperazinyl)-6-(trifluoromethyl)phenyl]methanol

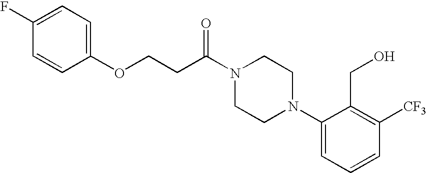

To a stirred solution of 2-(4-{3-[(4-fluorophenyl)oxy]propanoyl}-1-piperazinyl)-6-(trifluoromethyl)benzaldehyde (Compound 7) (25.5 mg, 0.06 mmol) in THF (3 mL) at 0° C. was added sodium borohydride (11.4 mg, 0.3 mmol). After 35 minutes the reaction mixture was poured into water (20 mL) and extracted with DCM (3×20 mL). The organic phases were combined and dried over Na₂SO₄ and the solvent removed. The residue was purified on silica gel eluting with 40-60% ethylacetate/hexane to afford the title compound (24 mg, 94%). Further purification was possible by recrystallisation from ethylacetate/hexane (1:1) to afford the title compound (14 mg); m/z: 427 [M+H]⁺, retention time 3.07 mins.

### Compound 17: 1-(2,3-Dichlorophenyl)-4-[3-(phenyloxy)propanoyl]piperazine

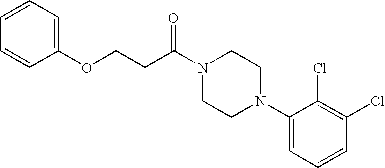

To a suspension of 3-(phenyloxy)propanoic acid (Aldrich) (0.62 g, 3.7 mmol), EDC hydrochloride (1.06 g, 5.5 mmol) and HOBt (0.5 g, 3.7 mmol) in dioxan (20 mL) at room temperature was added 2,3 dichlorophenyl piperazine (Fluorochem) (1.0 g, 3.7 mmol) and DIPEA (0.65 mL). The resulting solution was stirred at room temperature for 48 hrs and then concentrated. The residue was partitioned between ethyl acetate and water and the organic layer washed with 1N NaOH and then water. The organics were dried over Na₂SO₄, concentrated and then purified on silica eluting with 30% ethyl acetate/petrol (bp 40-60° C.) to afford the title compound as a white solid (1.03 g); m/z: 379/381 [M+H]⁺, retention time 3.49 mins.

### Compound 18: 2-(4-{3-[(3-Bromophenyl)oxy]propanoyl}-1-piperazinyl)-6-(trifluoromethyl)benzonitrile

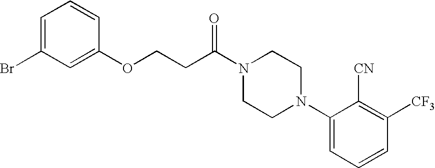

The title compound was prepared from 3-(3-bromophenoxy)propionic acid (Enamine) and 2-(1-piperazinyl)-6-(trifluoromethyl)benzonitrile (Intermediate 1) using a procedure similar to that described for the preparation of Compound 17; m/z: 482/484 [M+H]⁺, retention time 3.33 mins.

### Compound 19: 5-Chloro-2-(4-{3-[(4-fluorophenyl)oxy]propanoyl}-1-piperazinyl) benzonitrile

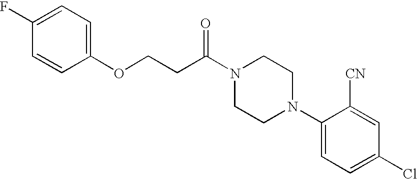

Saturated aqueous NaHCO₃ was added to 1-{3-[(4-fluorophenyl)oxy]propanoyl}piperazine hydrochloride (Intermediate 19) (37 mg, 0.13 mmol) to generate the free base and then extracted with DCM. The organic phases were combined and dried to give a colourless oil which was combined with 5-chloro-2-fluorobenzonitrile (20 mg, 0.13 mmol) in DMF (1 mL) and heated using a microwave reactor at 130° C. for 30 mins and then for an additional 10 mins at 150° C. The reaction mixture was passed through an SCX cartridge, eluting with DCM, MeOH and 2M NH₃ in MeOH. The mixture was concentrated in vacuo and the residue purified on silica eluting with 5% EtOAc/hexane to 50% EtOAc/hexane to afford the title compound as a white solid (3.5 mg); ¹H NMR (CDCl₃): 7.56-6.86 (7H, m), 4.30 (2H, m), 3.82 (4H, m), 3.18 (4H, m), 2.85 (2H, m).

### Compound 20: 2-Chloro-6-(4-{3-[(4-fluorophenyl)oxy]propanoyl}-1-piperazinyl)benzonitrile

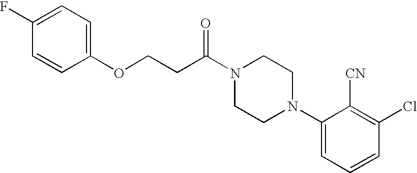

1-{3-[(4-fluorophenyl)oxy]propanoyl}piperazine hydrochloride (Intermediate 19) (50 mg, 0.17 mmol) and 2-chloro-6-fluorobenzonitrile (Aldrich) (27 mg, 0.17 mmol) were dissolved in DMF (1.5 mL) and DIPEA added (59 μL, 0.34 mmol). The mixture was heated in a microwave at 180° C. for 15 mins and then at 180° C. for 30 mins. The mixture was diluted with ethyl acetate and washed with ice-water. The organic phase was dried over Na₂SO₄ and concentrated to afford a brown oil which was purified on silica eluting with 10% ethylacetate/hexane—50% ethylacetate/hexane to afford the title compound as a white solid (8.2 mg); m/z: 388 [M+H]⁺, retention time 3.19 mins.

### Compound 21: 5-fluoro-2-{4-[3-(phenyloxy)propanoyl]-1-piperazinyl}benzonitrile

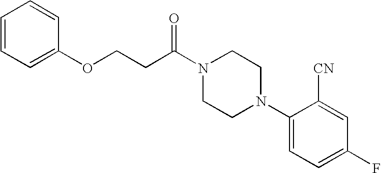

To polymer supported carbodiimide (Polymer Labs (194 mg, 0.25 mmol) was added HOAt (3.4 mg, 0.025 mmol) in THF/DCM (1:1, 2 mL). 3-Phenoxypropionic acid (Aldrich) (21 mg, 0.125 mmol) in THF (0.5 mL) and 5-fluoro-2-(1-piperazinyl)benzonitrile (Intermediate 12) (31 mg, 0.15 mmol) in DCM (0.5 mL) were added and the mixture shaken for 72 hrs at room temperature. Scavenger resins, MP-NCO (185 mg, 0.25 mmol) and MP-CO3 (94 mg, 0.25 mmol) (Biotage) were added. The mixture was stirred overnight, filtered and the resins washed with ethyl acetate, DCM and methanol. The organic phase was concentrated and the residue purified to afford the title compound (41.8 mg); ¹H NMR (CDCl₃): 7.28 (4H, m), 6.95 (4H, m), 4.35 (2H, m), 3.87 (2H, m), 3.77 (2H, m), 3.16 (2H, m), 3.09 (2H, m), 2.88 (2H, m). m/z: 354 [M+H]⁺, retention time 3.06 mins.

The following compounds of formula (Ia), i.e. compounds of general formula (I) where n is 0, were prepared by a similar procedure to that described for the preparation of Compound 21 using the appropriate piperazine intermediate or commercially available piperazine and appropriate commercially available carboxylic acid.

| | |
|---|---|
| | (Ia) |
| 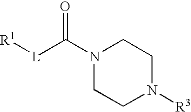 | |

| | | | | | Retention |
|---|---|---|---|---|---|
| | | | | | time (min) |
| | | | | m/z | 5 min |
| Cmp | R¹ | L | R³ | [M + H]⁺ | LC/MS |
| 22 | 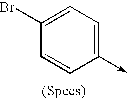 | —OCH₂CH₂— | 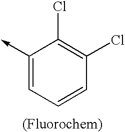 | 457, 459, 461, 463 | 3.64 |
| 23 | 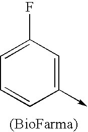 | —OCH₂CH₂— | 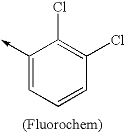 | 397, 399, 401 | 3.45 |
| 24 | 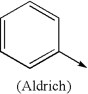 | —C(═O)CH₂CH₂— | 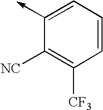 | 416 | 1.16 |

### Compound 25: 2-(4-{3-[(4-Fluorophenyl)oxy]propanoyl}-1-piperazinyl)benzonitrile

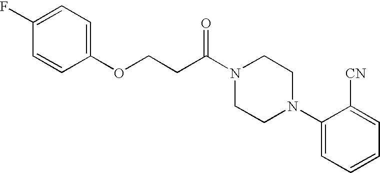

3-[(4-Fluorophenyl)oxy]propanoic acid (Aldrich) (0.05 mmol) was dissolved in THF/NMP (3:1, 250 μL) in a Robbins block (96 well reactor blocks available from Genetic Research Instrumentation). To this was added 2-(1-piperazinyl)benzonitrile (Aldrich) (0.06 mol) in DCM/THF (1:1, 250 μL), polymer supported EDC (0.1 mmol) and HOAt (0.01 mol) dissolved in DCM/THF (1:1, 0.8 mL). The Robbins blocks were shaken for 2 days and then polymer supported isocyanate (0.1 mmol) and polymer supported carbonate (0.1 mmol) were added followed by THF/DCM (600 μl of a 1:1 mixture). After shaking for a further 2 days the supported reagents were removed by filtration and washed with DCM/THF (3 mL of a 1:1 mixture). The organic solvents were removed to afford the title compound; m/z: 354 [M+H]⁺, retention time 3.33 mins.

The following compounds of formula (Ia), i.e. compounds of general formula (I) where n is 0, were prepared by a similar procedure to that described for the preparation of compound 25 using the appropriate commercially available piperazine and commercially available carboxylic acid.

| | |
|---|---|
| | (Ia) |
| 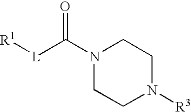 | |

| | | | | | Retention |
|---|---|---|---|---|---|
| | | | | m/z | time (min) |
| Cmp | R¹ | L | R³ | [M + H]⁺ | 5 min LC/MS |
| 26 | 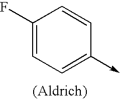 | —OCH₂CH₂— | 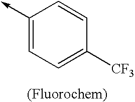 | 397 | 3.45 |
| 27 | 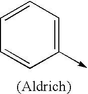 | —OCH₂CH₂— | 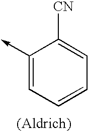 | 336 | 3.15 |
| 28 | 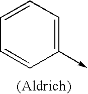 | —OCH₂CH₂— | 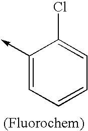 | 345/347 | 3.35 |
| 29 | 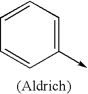 | —OCH₂CH₂— | 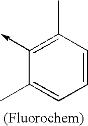 | 339 | 3.50 |
| 30 | 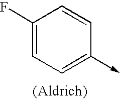 | —C(═O)CH₂CH₂— | 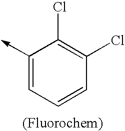 | 409 | 2.97 |
| 31 | 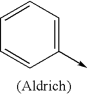 | —OCH₂CH₂— | 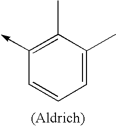 | 339 | 3.51 |

### Compound 32: 1-(2,3-Dichlorophenyl)-4-{3-[(3,4-difluorophenyl)oxy]propanoyl}piperazine

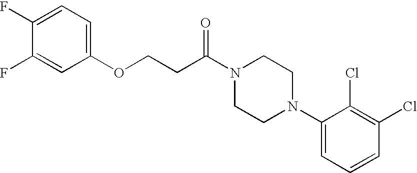

To a stirred solution of 1-(2,3-dichlorophenyl)piperazine (97 mg, 0.32 mmol), 3-[(3,4-difluorophenyl)oxy]propanoic acid (BioFarma) (65 mg, 0.32 mmol), HOAt (44 mg, 0.32 mmol) and DIPEA (0.16 mL, 0.96 mmol) in DCM (10 mL) was added HATU (115 mg, 0.32 mmol). The resulting mixture was stirred at room temperature for 14 hrs and then washed with brine and water, dried (MgSO₄) and then concentrated. The residue was purified by silica gel chromatography to afford the title compound (52 mg); m/z: 415/417/419 [M+H]⁺, retention time 3.59 mins.

### Compound 33: 2-(4-{3-[(3-Fluorophenyl)oxy]propanoyl}-1-piperazinyl)-6-(trifluoromethyl)benzonitrile

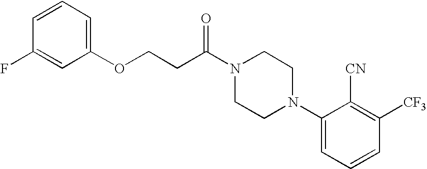

The title compound was prepared by a similar procedure to that described for compound 32 using 2-(1-piperazinyl)-6-(trifluoromethyl)benzonitrile (Intermediate 1) and 3-[(3-fluorophenyl)oxy]propanoic acid (BioFarma). m/z: 422 [M+H]⁺, retention time 3.30 mins.

### Biological Assay

### Primary Cortical Astrocyte Culture

Primary glial cultures were prepared from cortices of Sprague Dawley (CD1—cesarian derived) rats—postnatal day 2. The cortices were dissected and then dissociated by trituration in Dulbecco's Modified Eagle's Medium (D-MEM containing 1000 mg/L glucose, Invitrogen) supplemented with papain (30 U/mL, Worthington Laboratories), 0.24 mg/mL L-cysteine (Sigma), 40 μg/mL DNAse I type IV (Sigma). Papain treatment was stopped with D-MEM supplemented with 1 mg/mL trypsin inhibitor (Sigma), 50 μg/mL BSA (Sigma) and 40 μg/mL DNAse I type IV (Sigma). The resulting cell homogenate was plated onto T175 flasks—pre-coated with poly-D-lysine (MW: >300,000, Sigma) in D-MEM supplemented with 15% heat-inactivated fetal bovine serum (FBS), 2.2 mL glucose, penicillin and streptomycin and incubated at 37° C. and 5% CO₂. 3 days after plating the media was replaced with fresh growth medium (as above). 3 days later the medium was replaced with D-MEM supplemented with 10% heat-inactivated fetal bovine serum (FBS), 2.2 mL glucose, penicillin and streptomycin for 6 hours. The flasks were then shaken vigorously overnight at 37° C. to remove oligodendrocytes leaving astrocytes adhered to the flasks. The next day cells were subplated by trypinisation onto poly-D-lysine precoated 384-well plates at a density of 10,000 cells per well in D-MEM supplemented with 10% dialysed heat-inactivated fetal bovine serum (FBS), 2.2 ml glucose, penicillin and streptomycin and growth factors (basic fibroblast growth factor (5 ng/mL) and epidermal growth factor (10 ng/mL) incubated at 37° C. and 5% CO2 for 48 hours prior to assaying. Under exposure to growth factors rat cultured astrocytes express increased levels of mGlu5 receptors.

CHO Cells Expressing Human mGluR5b

CHO cells expressing human mGluR5b (pSwitch vector, Invitrogen) were maintained in DMEM media with 10% Foetal Calf Serum, 0.005% Hygromycin, Proline (10 mg/mL), Zeocin (0.005%).

The media was replaced between subculturing days to remove any excess glutamate build up that may cause receptor desensitization.

24 hrs prior to conducting the assay, cells were plated in 384-well black flat clear bottom plates and mGluR5 expression was induced with the addition of 0.01 nM mifepristone. The plates were incubated at 37° C. to give a monolayer with a confluency of 80%.

### FLIPR Assay

The tissue culture medium was aspirated using a Tecan power washer. Cells were loaded with 30 μl of Hanks Balanced Salts (HBSS)+2.5 M Probenicid+2 μM Fluo-4+250 μM Brilliant Black+0.01% Pluronic acid. The cells were incubated at 37° C. for 2 hours to allow uptake of the dye Fluo-4AM into the cell cytosol, which was converted to Fluo-4 by natural esterases cleaving the AM region preventing the Fluo-4 leaving the cell.

### Compound Preparation

Compounds to be tested were prepared by dissolving in DMSO at a concentration of 10 mM. These solutions were further diluted with DMSO using a Biomek FX (Beckman Coulter) in a 384 well compound plate. Each dilution was transferred in 1 μL aliquots to a further compound plate and assay buffer (HBSS+2.5 M Probenicid) was added to a volume of 50 μL, making a final assay concentration of 11.8 μM in the assay.

### Estimation of ˜EC20 Concentration of Glutamate (Endogenous Ligand)

A 0.38 M solution of glutamic acid was prepared in water. This was further diluted in DMSO to a concentration of 255 mM. A range of concentrations of glutamate were prepared in assay buffer and dispensed using a Multidrop (Thermolabsystems) into a 384 well compound plate. The chosen standard positive modulator was prepared in a 384 well compound plate at a concentration of 1 mM in DMSO. A Biomek FX was used to produce a serial dilution in DMSO. 1 μL of each dilution was transferred to a further compound plate where it was diluted in 50 μL of assay buffer. This gives a final top assay concentration of 4 μM. 10 μL of this compound plate was added ‘on line’ on FLIPR to a loaded cell plate. A fluorescent reading was made. The cells with compound added were incubated at 37° C. for 10 min after which a plate containing the range of glutamate concentrations was added to the cells in a second ‘on line’ FLIPR addition. A second fluorescent read was made. A visual choice of EC20 concentration was made from the raw data generated. The chosen concentration of glutamate was dispensed into 384 well compound plates using a Multidrop and was high enough to allow modulation to be viewed without being so high that it masked modulation.

### Running the Assay

The compounds were added ‘on line’ to the cells and a first FLIPR read was made to estimate agonism. The cell plates were then incubated at 37° C. for 5-10 min after which the chosen EC20 Glutamate was added ‘on line’ and a second FLIPR reading made to estimate modulation. Modulation potency was normalised to EC20 Glutamate response as basal and EC100 Glutamate response as total.

Supporting compounds 1-33 showed activity in the human mGluR5b positive modulator assay with a pEC50 value range from 5.8 to 7.1

1. A compound of formula (I) or a pharmaceutically acceptable salt thereof 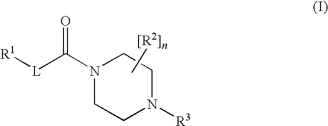
wherein
R¹ is phenyl optionally substituted by one or more halogens;
L is a three atom linker selected from the group consisting of
—OC(R^{a})₂C(R^{b})₂—, and —C(═O)C(R^{a})₂C(R^{b})₂—, wherein the left hand atom of the linker is attached to R¹;
wherein each R^{a} which may be the same or different, is hydrogen or C₁₋₃alkyl; or the two R^{a} groups together with the carbon atom to which they are attached form a 3-6-membered cycloalkane ring;
each R^{b} which may be the same or different, is hydrogen or C₁₋₃alkyl; or
the two R^{b} groups together with the carbon atom to which they are attached form a 3-6-membered cycloalkane ring; and
wherein either the two R^{a} groups or the two R^{b} groups may form a cycloalkane ring;
n is 0, 1 or 2;
when n is 1 or 2, R² is independently C₁₋₃alkyl or haloC₁₋₃alkyl; and
R³ is phenyl optionally substituted by one or more groups independently selected from the group consisting of halogen, C₁₋₃alkyl, haloC₁₋₃alkyl, cyano, —CH(═NOH) and hydroxyC₁₋₃alkyl; wherein one of the groups is attached to the ortho position on the phenyl;
wherein the compound is not 4-oxo-1-phenyl-4-(4-phenyl-1-piperazinyl)-1-butanone;
1-(4-fluorophenyl)-4-oxo-4-(4-phenyl-1-piperazinyl)-1-butanone;
1-(4-fluorophenyl)-4-[4-(2-fluorophenyl)-1-piperazinyl]-4-oxo-1-butanone;
4-[4-(5-chloro-2-methylphenyl)-1-piperazinyl]-4-oxo-1-phenyl-1-butanone;
4-[4-(2-chlorophenyl)-1-piperazinyl]-4-oxo-1-phenyl-1-butanone; or
1-(2-methylphenyl)-4-[3-(phenyloxy)propanoyl]piperazine.

2. The compound according to claim 1 or a pharmaceutically acceptable salt thereof, wherein, L is a three atom linker selected from the group consisting of —OC(R^{a})₂C(R^{b})₂— and —C(═O)C(R^{a})₂C(R^{b})₂—, wherein the left hand atom of the linker is attached to R¹, wherein each R^{a} which may be the same or different, is hydrogen or C₁₋₃alkyl; and each R^{b} which may be the same or different, is hydrogen or C₁₋₃alkyl.

3. The compound according to claim 1 or a pharmaceutically acceptable salt thereof, wherein R¹ is phenyl optionally substituted by one or more halogens;
L is —OC(R^{a})₂C(R^{b})₂—, wherein the left hand atom of the linker is attached to R¹;
wherein each R^{a} which may be the same or different, is hydrogen or C₁₋₃alkyl; or the two R^{a} groups together with the carbon atom to which they are attached form a 3-6-membered cycloalkane ring;
each R^{b} which may be the same or different, is hydrogen or C₁₋₃alkyl; or
the two R^{b} groups together with the carbon atom to which they are attached form a 3-6-membered cycloalkane ring; and
wherein either the two R^{a} groups or the two R^{b} groups may form a cycloalkane ring;
n is 0, 1 or 2;
when n is 1 or 2, R² is independently C₁₋₃alkyl or haloC₁₋₃alkyl; and
R³ is phenyl optionally substituted by one or more groups independently selected from the group consisting of halogen, C₁₋₃alkyl, haloC₁₋₃alkyl, cyano, —CH(═NOH) and hydroxyC₁₋₃alkyl; wherein one of the groups is attached to the ortho position on the phenyl;
wherein the compound is not 1-(2-methylphenyl)-4-[3-(phenyloxy)propanoyl]piperazine.

4. The compound according to claim 1 or a pharmaceutically acceptable salt thereof, wherein L is —OCH₂CH₂—, wherein the left hand atom of the linker is attached to R¹.

5. The compound according to claim 1 or a pharmaceutically acceptable salt thereof, wherein n is 0.

6. The compound according to claim 1 or a pharmaceutically acceptable salt thereof, wherein R³ is phenyl substituted by one, two or three groups independently selected from the group consisting of halogen, C₁₋₃alkyl, haloC₁₋₃alkyl, cyano, —CH(═NOH) and hydroxyC₁₋₃alkyl; one group being substituted at the ortho position by either cyano, —CH(═NOH) or hydroxyC₁₋₃alkyl.

7. The compound according to claim 1 or a pharmaceutically acceptable salt thereof, wherein R³ is phenyl substituted by one or two groups independently selected from the list consisting of halogen, C₁₋₃alkyl, haloC₁₋₃alkyl, cyano, —CH(═NOH) and hydroxyC₁₋₃alkyl; one group being substituted at the ortho position by cyano.

8. A compound selected from the group consisting of:
2-(4-{3-[(4-fluorophenyl)oxy]propanoyl}-1-piperazinyl)-6-(trifluoromethyl) benzonitrile;
2,3-dichloro-6-(4-{3-[(4-fluorophenyl)oxy]propanoyl}-1-piperazinyl)benzonitrile;
2-(4-{3-[(4-fluorophenyl)oxy]propanoyl}-1-piperazinyl)-5-(trifluoromethyl)benzonitrile,
3-chloro-6-(4-{3-[(4-fluorophenyl)oxy]propanoyl}-1-piperazinyl)-2-(trifluoromethyl)benzonitrile;
1-(2,3-dichloro-4-fluorophenyl)-4-{3-[(4-fluorophenyl)oxy]propanoyl}piperazine;
2-chloro-3-fluoro-6-(4-{3-[(4-fluorophenyl)oxy]propanoyl}-1-piperazinyl)benzonitrile;
2-(4-{3-[(4-fluorophenyl)oxy]propanoyl}-1-piperazinyl)-6-(trifluoromethyl)benzaldehyde;
2-fluoro-6-(4-{3-[(4-fluorophenyl)oxy]propanoyl}-1-piperazinyl)benzonitrile;
3,5-dichloro-2-(4-{3-[(4-fluorophenyl)oxy]propanoyl}-1-piperazinyl)benzonitrile;
1-[2-chloro-3-(trifluoromethyl)phenyl]-4-{3-[(4-fluorophenyl)oxy]propanoyl}piperazine;
2-{4-[3-(phenyloxy)propanoyl]-1-piperazinyl}-6-(trifluoromethyl)benzonitrile;
1-(2,3-dichlorophenyl)-4-{3-[(4-fluorophenyl)oxy]propanoyl}piperazine,
5-fluoro-2-(4-{3-[(4-fluorophenyl)oxy]propanoyl}-1-piperazinyl)benzonitrile;
2-(4-{3-[(4-fluorophenyl)oxy]propanoyl}-1-piperazinyl)-6-(trifluoromethyl)benzaldehyde oxime;
2-(4-{3-[(4-fluorophenyl)oxy]propanoyl}-1-piperazinyl)-6-(trifluoromethyl)benzaldehyde O-methyloxime;
[2-(4-{3-[(4-fluorophenyl)oxy]propanoyl}-1-piperazinyl)-6-(trifluoromethyl)phenyl]methanol;
1-(2,3-dichlorophenyl)-4-[3-(phenyloxy)propanoyl]piperazine;
2-(4-{3-[(3-bromophenyl)oxy]propanoyl}-1-piperazinyl)-6-(trifluoromethyl)benzonitrile;
5-chloro-2-(4-{3-[(4-fluorophenyl)oxy]propanoyl}-1-piperazinyl)benzonitrile;
2-chloro-6-(4-{3-[(4-fluorophenyl)oxy]propanoyl}-1-piperazinyl)benzonitrile;
5-fluoro-2-{-4-[3-(phenyloxy)propanoyl]-1-piperazinyl}benzonitrile;
1-{3-[(4-bromophenyl)oxy]propanoyl}-4-(2,3-dichlorophenyl)piperazine;
1-(2,3-dichlorophenyl)-4-{3-[(3-fluorophenyl)oxy]propanoyl}piperazine;
2-[4-(4-oxo-4-phenylbutanoyl)-1-piperazinyl]-6-(trifluoromethyl)benzonitrile;
2-(4-{3-[(4-fluorophenyl)oxy]propanoyl}-1-piperazinyl)benzonitrile;
1-{3-[(4-fluorophenyl)oxy]propanoyl}-4-[4-(trifluoromethyl)phenyl]piperazine;
2-{4-[3-(phenyloxy)propanoyl]-1-piperazinyl}benzonitrile;
1-(2-chlorophenyl)-4-[3-(phenyloxy)propanoyl]piperazine;
1-(2,6-dimethylphenyl)-4-[3-(phenyloxy)propanoyl]piperazine;
4-[4-(2,3-dichlorophenyl)-1-piperazinyl]-1-(4-fluorophenyl)-4-oxo-1-butanone;
1-(2,3-dimethylphenyl)-4-[3-(phenyloxy)propanoyl]piperazine;
1-(2,3-dichlorophenyl)-4-{3-[(3,4-difluorophenyl)oxy]propanoyl}piperazine; and
2-(4-{3-[(3-fluorophenyl)oxy]propanoyl}-1-piperazinyl)-6-(trifluoromethyl)benzonitrile;
or a pharmaceutically acceptable salt thereof.

9.-13. (canceled)

14. A pharmaceutical composition comprising a compound as defined in claim 1 or a pharmaceutically acceptable salt thereof.

15. A method for the treatment of diseases or conditions mediated by positive allosteric modulation of mGluR5 comprising administering to a subject the compound according to claim 1.

16. The compound according to according to claim 15 or a pharmaceutically acceptable salt thereof, wherein the disease or condition is a psychotic disorder.

17. The compound according to according to claim 16 or a pharmaceutically acceptable salt thereof, wherein the psychotic disorder is schizophrenia.
